(19) United States
(12) Patent Application Publication
(10) Pub. No.: US 2022/0386984 A1
(43) Pub. Date: Dec. 8, 2022

(54) APPARATUS FOR DETECTING BREATH SOUNDS

(71) Applicant: Respiri Limited, Melbourne, Victoria (AU)

(72) Inventors: Samaneh Sarraf, Melbourne, Victoria (AU); Adam Ratcliff, Melbourne, Victoria (AU); Nathan Maina, Melbourne, Victoria (AU); Mike Smith, Melbourne, Victoria (AU)

(21) Appl. No.: 17/755,485

(22) PCT Filed: Oct. 29, 2020
(86) PCT No.: PCT/AU2020/051173

§ 371 Date: Apr. 29, 2022

(30) Foreign Application Priority Data
Oct. 29, 2019 (AU) 2019904065

(51) Int. Cl. A61B 7/00; B81B 3/00; H04R 1/46; H04R 1/08; H04R 1/28; H04R 1/02; A61B 5/08

(52) U.S. Cl. CPC A61B 7/003; B81B 3/0021; H04R 1/46; H04R 1/083; H04R 1/28; H04R 1/02; A61B 5/08; H04R 2201/003; B81B 2201/0257; A61B 2562/028

(57) ABSTRACT

A contact sensor for monitoring breathing of a subject, comprising: a microphone housing defining a first acoustic cavity, a MEMS microphone disposed within the first acoustic cavity; a second acoustic cavity separated from the first acoustic cavity by a cavity wall having a front surface and a rear surface, the second acoustic cavity at least partially defined by the front surface of the cavity wall; an acoustic conduit formed between the first acoustic cavity and the second acoustic cavity through the cavity wall; and a pressure relief vent having a first end terminating at the second acoustic cavity and a second end terminating outside of the second acoustic cavity.

### TECHNICAL FIELD

The present disclosure relates to contact sensors in particular contact microphones for detecting breath sounds.

### BACKGROUND

Asthma is a long-term inflammatory disease of the airways of the lungs, causing variable and reoccurring symptoms of wheezing, coughing and shortness of breath. A conventional device used to monitor asthmatic symptoms is a peak flow meter; a handheld device which measures a person's maximum speed of expiration and thus the degree of obstruction in their airways. The effectiveness of a peak flow monitor for self-diagnosis of symptoms is, however, limited, due to the wide range of ‘normal’ values of peak flow and the high degree of variability in results.

Wheeze monitors, such as the Airsonea® by iSonea®, have been developed which monitor vibrations of the trachea using a contact sensor placed on the skin in proximity to the trachea. Such devices use piezoelectric sensors to pick up breath sounds which can be analysed to determine whether the patient is wheezing and to what degree.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

According to a first aspect of the disclosure, there is provided a contact sensor for monitoring breathing of a subject, comprising: a microphone housing defining a first acoustic cavity, a MEMS microphone disposed within the first acoustic cavity; a second acoustic cavity separated from the first acoustic cavity by a cavity wall having a front surface and a rear surface, the second acoustic cavity at least partially defined by the front surface of the cavity wall; an acoustic conduit formed between the first acoustic cavity and the second acoustic cavity through the cavity wall; and a pressure relief vent having a first end terminating at the second acoustic cavity and a second end terminating outside of the second acoustic cavity.

In some embodiments, the contact sensor may further comprise a flexible membrane formed over the cavity wall, the flexible membrane having a front surface and a rear surface facing the front surface of the cavity wall.

The contact microphone may further comprise a gasket between the rear surface of the flexible diaphragm and the front surface of the cavity wall extending around an outer limit of the cavity wall, wherein the pressure relief vent comprises a notch formed in the gasket.

The flexible membrane may comprise silicone or biaxially-oriented polyethylene terephthalate (Mylar®) or glass-reinforced epoxy laminate. The flexible membrane may have a thickness of between 0.4 mm and 0.8 mm or a thickness of approximately 0.55 mm to 0.65 mm, particularly when made from silcone. The flexible membrane may has a thickness of approximately 0.1 mm, for example when made from glass-reinforced epoxy laminate, or 0.07 mm, for example when made from Mylar.

The flexible membrane may have a Shore durometer as measured using ASTM D2240 type A of between 60 and 80. For example, the flexible membrane may a Shore durometer as measured using ASTM D2240 type A of approximately 73.

In some embodiments, the cavity wall may comprise a contact surface extending around an outer limit of the front surface cavity wall, the flexible membrane partially or completely omitted. In use, the contact surface may be configured to contact the surface of the subject. The cavity wall and the surface of the subject may then form an acoustic chamber.

In either case, the pressure relief vent may be configured to vent air between the second acoustic cavity and the atmosphere. The pressure relief vent may comprise a notch formed in the front surface of the cavity wall terminating at an outer limit of the cavity wall. In some embodiments, the flexible membrane is part of a cover, and wherein the pressure relief vent further comprises a side vent in the cover in fluid communication with the notch. Alternatively, the pressure relief vent may comprise a passage formed between the front surface of the cavity wall and the rear surface of the cavity wall.

The contact sensor may further comprise a membrane filter disposed proximate the second end of the vent. The membrane filter may comprise expanded polytetrafluoroethylene (ePTFE).

The front surface of the cavity wall may have a bowl shape or a horn shape for focusing pressure waves towards an entrance of the conduit in the cavity wall.

The acoustic conduit may terminate at a location approximately at the centre of the front surface of the cavity wall. The acoustic conduit may have a diameter of approximately 0.5 mm. The acoustic conduit may have a length of between 0.5 mm and 5.0 mm.

The contact sensor may further comprise a damping mass coupled to the microphone housing. The damping mass may comprise aluminium or stainless steel.

The contact sensor may further comprise a printed circuit board (PCB). In which case, the MEMS microphone may be mounted on the PCB.

The rear surface of the cavity wall may comprise a plurality of pins extending therefrom. The PCB may comprise a plurality apertures formed therethrough, each of the plurality of pins configured to engage with a respective aperture of the plurality of apertures so as to align the microphone housing, for example with the acoustic conduit. Engagement of the plurality of pins with the plurality of apertures may align an acoustic aperture in the microphone housing with the acoustic conduit. The plurality of pins may be heat staking pins. In which case, the PCB may be fixed relative to the cavity wall by the plurality of pins.

A second gasket may be provided between the microphone housing and the rear surface of the cavity wall. The second gasket may extend around an outer limit of the acoustic aperture.

According to a further aspect of the disclosure, there is provided a device, comprising an enclosure; the contact microphone of any one of the preceding claims; and a background microphone configured to receive ambient sound, wherein the contact microphone and the background microphone housed in the enclosure.

The MEMS microphone and the background microphone may be substantially acoustically decoupled.

The device may further comprise one or more acoustic dampeners housed in the enclosure.

The enclosure may have a first end and a second end opposite the first end. The contact surface of the flexible diaphragm, when provided, may be located proximate the first end of the enclosure. The background microphone may be located proximate the second end of the enclosure.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

Embodiments of the present disclosure will now be described by way of non-limiting examples, with reference to accompanying figures, in which:

FIG. 1 is a cross-section diagram of an apparatus according to an embodiment of the present disclosure;

FIG. 2 is a perspective exploded view of the apparatus shown in FIG. 1;

FIG. 3 is a close up cross-sectional view of a MEMS microphone of the apparatus shown in FIG. 1;

FIG. 4 is a graph comparing signals acquired by the MEMS microphone of the apparatus shown in FIG. 1 with various vent configurations;

FIG. 5 is a graph showing the frequency response of signal acquired by the MEMS microphone of the apparatus shown in FIG. 1 with various vent configurations;

FIG. 6 is a cross-section view of a cavity wall of the apparatus of FIG. 1 having a bowl shaped front surface;

FIG. 7 is a cross-section view of a cavity wall of the apparatus of FIG. 1 having a concave horn shaped front surface;

FIG. 8 is a cross-section view of a cavity wall of the apparatus of FIG. 1 having a convex horn shaped front surface;

FIG. 9 is a perspective view of a device comprising the apparatus shown in FIG. 1;

FIG. 10 is a cross-section view of the device shown in FIG. 9;

FIG. 11A is a cross-section diagram of an apparatus according to another embodiment of the present disclosure; and

FIG. 11B is the cross-section of FIG. 11A marked to show the passage of air flow.

### DESCRIPTION OF EMBODIMENTS

Embodiments of the present disclosure provide improvements the monitoring of breathing using contact sensors.

Embodiments of the present disclosure relate to contact sensors for sensing vibrations of animal or human anatomy due to breathing. Specifically, contact sensors are provided which use a MEMS microphone configured to translate vibrations of either the skin of a subject (optionally via a flexible diaphragm mechanically coupled to the skin) into electrical signals.

MEMS microphones are typically designed to function as conventional microphones, converting incident acoustic waves into electrical representations. When used for their conventional purpose, MEMS microphones exhibit wide and relatively flat frequency response and a high degree of linearity. However, MEMS microphones are not conventionally used in contact sensors, such as contact microphones where piezoelectric sensors are traditionally used because they do not require access to a column of air. The inventors have devised techniques to exploit the excellent sound capturing characteristics of MEMS microphones for contact sensing applications.

Embodiments of the present disclosure use an acoustic air-gap coupling to interface the skin (or a flexible diaphragm on the skin) with a MEMS microphone (which measures sound pressure/air pressure). As will be described in more detail below, the inventors have found that the quality of the acoustic signal acquired by MEMS microphone provided in this configuration is substantially affected by the level of venting of the acoustic air-gap. By providing venting, a flatter frequency response over a wider frequency range can be acquired with a reduction in low-frequency booming when compared to non-vented devices.

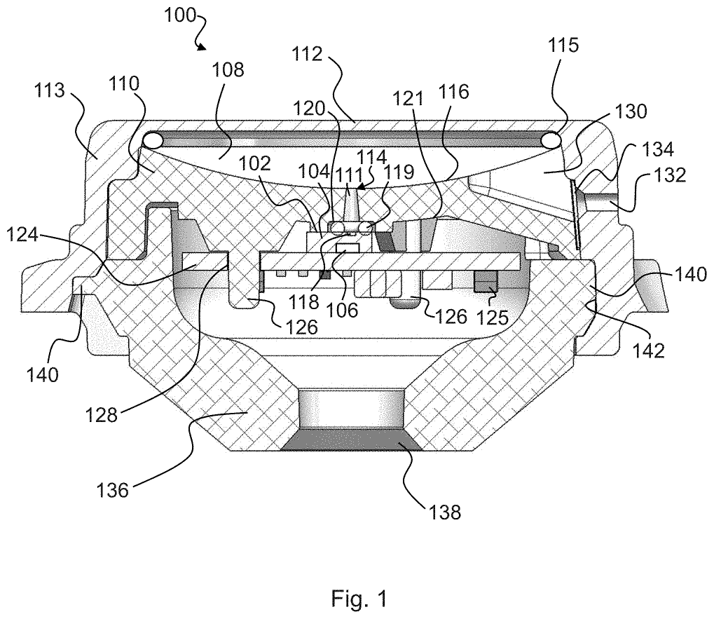

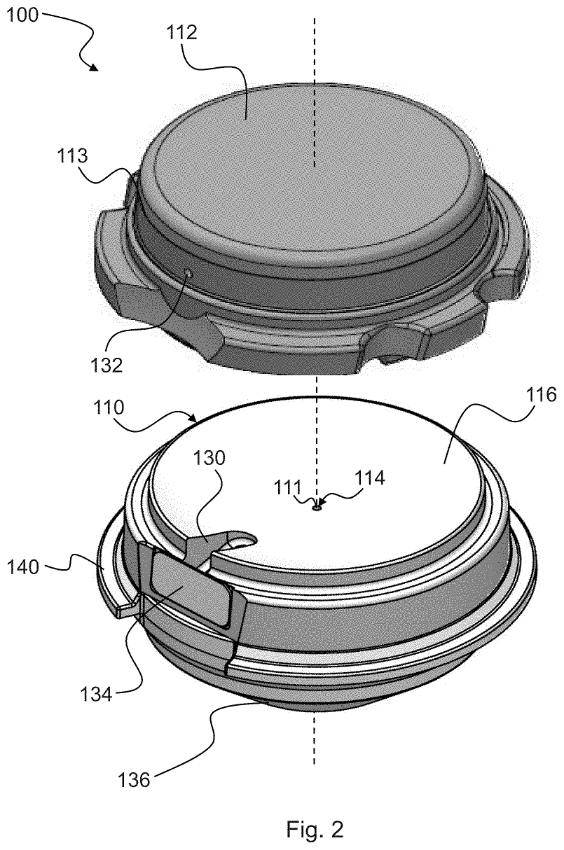

FIGS. 1 and 2 are cross-sectional and perspective exploded views of an apparatus 100 according to various embodiments of the present disclosure. The apparatus 100 comprises a microphone housing 102 defining a first acoustic cavity 104 which houses a MEMS microphone 106. The apparatus 100 further comprises a second acoustic cavity 108 defined by a cavity wall 110, which separates the second acoustic cavity 108 from the microphone housing 102.

In the embodiment of FIGS. 1 and 2, the apparatus 100 further comprises a diaphragm in the form of flexible membrane 112 disposed over cavity wall 110 to prevent ingress of dirt, water and other matter which may be detrimental to the proper functioning of the apparatus 100. In the embodiment shown, the flexible membrane 112 is formed as part of a cover 113 which extends over an outer limit of the cavity wall 110. In other embodiments, the flexible membrane 112 may be formed as a separate element. In yet further embodiments, the flexible membrane 112 may be omitted altogether, such that, in use, the surface of a subject (e.g. skin) forms a direct seal with an outer limit of the cavity wall 110 and thus acts as the flexible membrane 112. The functioning of such a device is this similar to that described below with the flexible membrane 112.

In the illustrated embodiment, a gasket 115 (e.g. an O-ring) is provided between the flexible membrane 112 and cavity wall 110 extending around the outer limit of the cavity wall 110 to provide a seal between the flexible membrane 112 and the cavity wall 110. The gasket 115 may also increase the volume of the second acoustic cavity 108. By varying the thickness of the gasket 115, the volume of the second acoustic cavity 108, and therefore the acoustic response of the second acoustic cavity 108, can be modified. In other embodiments, the gasket 115 may be omitted, the flexible membrane 112 and the cavity wall 110 forming a seal therebetween around the outer limit of the cavity wall 110.

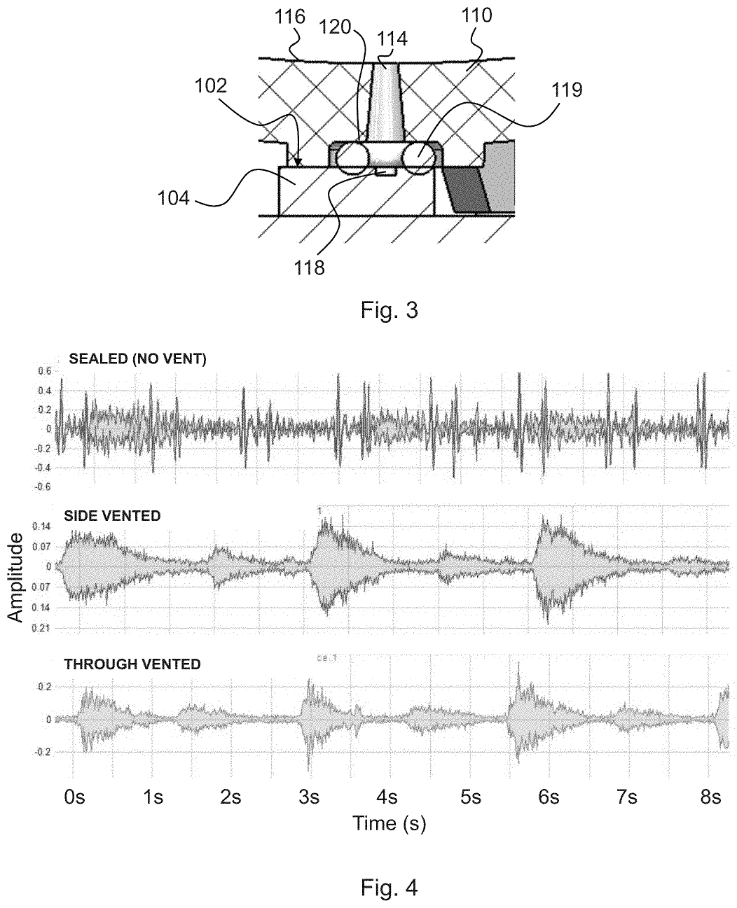

FIG. 3 is a close up cross-sectional view of a portion of the apparatus 100 in the vicinity of the microphone housing 102. As shown in FIG. 3, an acoustic conduit 114 is provided between the first acoustic cavity 104 and the second acoustic cavity 108 extending from the front surface 116 of the cavity wall 110 through an aperture 111 in the cavity wall 110 and through an aperture 118 formed in the microphone housing 102. The acoustic conduit 114 thereby allows acoustic waves to travel between the first acoustic cavity 104 and the second acoustic cavity 108. In the illustrated embodiment, a gasket 119 is provided between a rear surface 120 of the cavity wall 110 and the microphone housing extending around the perimeter of the acoustic conduit 114 to seal the coupling between the cavity wall 110 and microphone housing. In the embodiment shown, the rear surface 120 of the cavity wall 110 is provided with a recess configured to receive the gasket 119 (in this example, an o-ring), the gasket 119 being sized so as to elastically deform when the microphone housing 102 is brought into contact with the rear surface 121 of the cavity wall 110 during assembly and thus form a seal. The gasket 119 not only seals the acoustic conduit 114 at the coupling between the microphone housing 102 and the cavity wall 110 but additionally ensures a substantially consistent length of the acoustic conduit 114.

In use, movement of the flexible membrane 112 (or the skin or surface of a subject where the flexible membrane 112 is omitted) in a direction perpendicular to its surface, generates acoustic waves which travel from the flexible membrane 112 through the second acoustic cavity 108 and the acoustic conduit 114 to the first acoustic cavity 104 formed in the microphone housing 102, the acoustic waves picked up by the MEMS microphone 106.

The MEMS microphone 106 and/or microphone housing 102 may be mounted on a printed circuit board (PCB) 124 upon which may be provided circuitry 125, such as a digital signal processor (DSP), for processing signals acquired by the MEMS microphone 106. Such circuitry is well known in the art and so will not be described in detail here.

The rear surface of the cavity wall 110 may be provided with a plurality of pins 126 extending therefrom and the PCB 124 may be provided with a plurality of corresponding apertures 128 formed therethrough to fix the position of the PCB 124 relative to the cavity wall 110 so as to collocate the aperture 118 of the microphone housing 118 with the rear entry of the passage 111 formed in the cavity wall 110 during assembly. Each of the plurality of pins 126 may be configured to engage with a respective aperture of the plurality of apertures 128 so as to align the aperture 118 in the microphone housing 118 with the passage 111. In some embodiments, the plurality of pins 126 may be heat staking pins which may be configured to deform in response to being heated so as to fix the PCB 124 into a connected configuration with the cavity wall 110 as is shown in FIG. 1.

In other embodiments, the microphone housing 102 may be fixed directly to the cavity wall 110 or may be integrated into the cavity wall 110. In which case the gasket 119 and recess 122 may be omitted, and the acoustic conduit 114 formed as a single passage between the first and second acoustic cavities 104, 108.

As mentioned above, venting of the second acoustic cavity has been found to substantially improve the signal acquired by the MEMS microphone. Accordingly, in the illustrated embodiment, the apparatus 100 comprises a pressure relief vent extending between the second acoustic cavity 108 and the outside of the apparatus 100. In this example, the pressure relief vent is formed from a cut or notch 132 in the front surface 116 of the cavity wall 110 terminating at an outer limit of the cavity wall and a side vent 132 in the cover 113. The notch 132 and the side vent form an air passage between the second acoustic cavity 108 and the atmosphere which bypasses the top contact surface of the flexible membrane 112, allowing air to vent through the side of the apparatus 100 even when the top contact surface of the flexible membrane 112 is in contact with a measurement surface, such as the skin of a patient/user. As will be apparent from FIG. 2, in an example, where there is no cover, notch 132 would be sufficient to form a pressure relief vent that would provide an air passage between the second acoustic cavity 108 and the atmosphere.

In other embodiments, the gasket 115 (if provided) may comprise a break or notch forming an air passage between the second acoustic cavity 108 and the exterior of the apparatus 100.

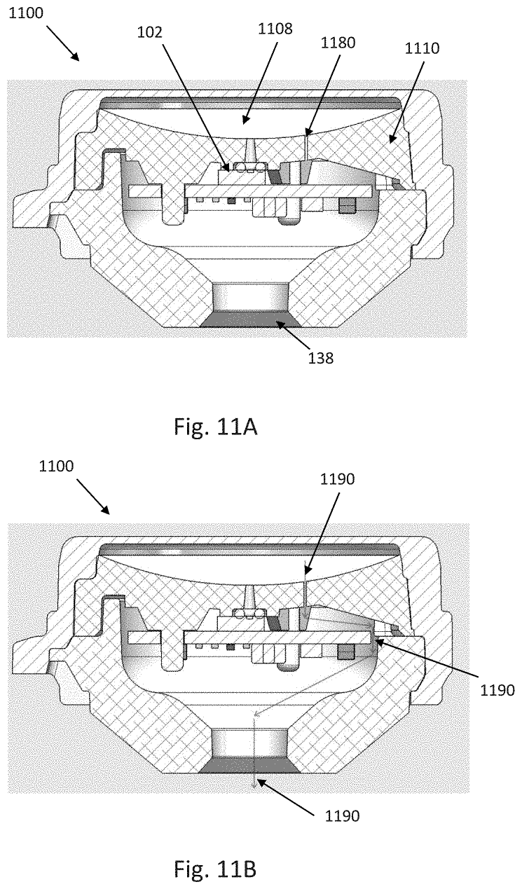

In yet other embodiments, instead of venting to the side of the apparatus 100, the pressure relief vent 130 may comprise an air passage formed through the cavity wall between the front surface 116 and a rear surface of the cavity wall 110. Such through venting may be provided instead of or in combination with side venting, such as that described above. A through vent is illustrated in FIG. 11A which is described below. When the apparatus 100 is incorporated into a device comprising multiple microphones, as will be described in more detail below, it may be preferred to implement the side vent configuration shown in FIG. 1 rather than venting to the rear surface 121 of the cavity wall, so as to reduce possible coupling of venting sounds to other microphones in proximity to the rear surface 121 of the cavity wall 110.

To prevent ingress of dirt and/or moisture into the second acoustic cavity 109 via the pressure relief vent 130, a filter 134 may be provided, disposed in the fluid path of the vent 130. The filter 134 may comprise a mesh, which may be substantially waterproof. For example, the filter 134 may comprise expanded polytetrafluoroethylene (ePTFE), e.g. Gore-Tex®.

The apparatus 100 further comprises a damping mass 136 provided to reduce the amplitude of mechanical vibrations in the apparatus 100.

Additionally, when integrated into a larger device, such as the device described below, the damping mass 136 reduces acoustic coupling between the MEMS microphone 106 and other microphone(s) in such devices. The damping mass 136 is preferably manufactured from aluminium to minimize the overall mass of the apparatus 100. Alternatively, the damping mass 136 may be stainless steel.

The damping mass 136 is coupled to the cavity wall 110 and therefore indirectly with the flexible membrane 112, the microphone housing 102 and the PCB 124. Optionally, a port 138 may be provided in the damping mass 136 distal from the cavity wall 110 to enable one or more cables (not shown), such as cable coupled to the PCB 124, to exit the apparatus 100, as will be described in more detail below. The damping mass 136 may be provided with a flange 140 extending around an outer limit of the damping mass 136 which may engage with an internal groove 142 in the cover 113 so as to secure the cover 113, and therefore the flexible membrane 114 relative to the damping mass 136 and the cavity wall 110. The cover 113 and the damping mass 136 may therefore form a captive or substantially sealed unit. In this regard, the apparatus 100 may be substantially sealed, the cover 113 being irremovable by a user or patient from the apparatus 100, to reduce risk of choking or other catastrophic risk from otherwise removable elements of the apparatus 100.

The provision of venting to/from the second acoustic cavity 108 allows excess air pressure created during diaphragm movements to pass outside of the second acoustic cavity 108. Such venting has been found to improve the quality of signals acquired by the MEMS microphone 106 in response to movement of the diaphragm 112.

FIG. 4 is a graph showing the breathing sounds acquired from the same subject by the MEMS microphone 106 of the apparatus 100 shown in FIG. 1 comprising the side vent 112 (middle), and similar apparatus to the apparatus 100 of FIG. 1 without venting (top) and with through venting (bottom). It can be seen that in absence of venting, significant noise is present in the acquired breath signal. In contrast, venting of the second acoustic cavity 108 leads to clear acquisition of the breath signal.

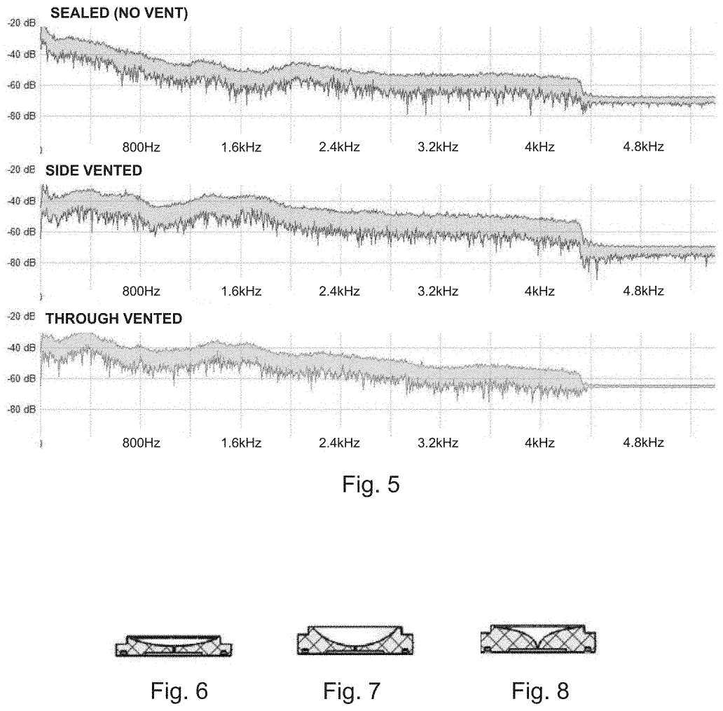

FIG. 5 is a graph comparing the frequency response of the apparatus 100 shown in FIG. 1 comprising the side vent 112 (middle), and similar apparatus to the apparatus 100 of FIG. 1 without venting (top) and with through venting (bottom). It can be seen from FIG. 5 that low frequency components of the signal acquired from the apparatus having no venting are unduly amplified. This low frequency gain or “booming” causes the MEMS microphone to saturate earlier, i.e. in response to smaller diaphragm movements when compared to the vented apparatus 100 (both side vented and through vented). Further, it can be seen that the frequency response of the side-vented apparatus 100 is flatter over a larger frequency range (e.g. 2 kHz to 4.5 KHz) than the through-vented apparatus.

In addition to venting, multiple other factors may affect transmission of sound between the upper surface of the flexible membrane 112 (or the surface of the subject if the membrane 112 is omitted) and the MEMS microphone 104, including but not limited to the material characteristics of the flexible membrane 112 (or surface of the subject), the shape and size of the second acoustic cavity 108, the shape and size of the acoustic conduit 114 and the shape and size of the first acoustic cavity 104.

In some embodiments, the front surface 116 of the cavity wall 110 may be shaped so as to increase the coupling of acoustic waves into the acoustic conduit 114. For example, the front surface of the cavity wall 110 may have a shallow bowl shape such as that in FIG. 6, a deeper bowl or concave horn shape such as that shown in FIG. 7 (FIG. 7 being similar to the cavity shape of FIG. 1), or a convex horn shape, such as that shown in FIG. 8. The acoustic conduit 114 may be located approximately at the centre of the horn or bowl or the centre of the cavity wall 110. It has been found that a cavity wall 110 having a concave horn shape such as shown in FIG. 7 leads to an improved frequency response and reduced reverberation time in signals output by the MEMS microphone 106 when compared with those shown in FIGS. 6 and 8.

In some embodiments, the acoustic conduit may have a diameter of between 0.4 mm and 0.6 mm, for example 0.5 mm. In some embodiments, the acoustic conduit may have a length of between 0.5 mm and 5.0 mm, or between 1.5 mm and 2.0 mm, for example 1.85 mm.

The flexible membrane 112 may be moulded as a single piece, for example as a single piece integrated with the cover 113.

For medical uses, the flexible membrane is preferably manufactured from a material that is both biocompatible and provides a suitable transfer media for coupling sound from a patient or user's skin to the second acoustic cavity 108. In some embodiments, the flexible membrane 112 may be formed from silicone, a thermoplastic polymer, such as polystyrene (ABS polymer), polypropylene, or polyethylene, a biaxially-oriented polyethylene terephthalate (e.g. Mylar®) or a glass-reinforced epoxy laminate (e.g. FR4). The inventors have found that the use of Mylar for the flexible membrane 112 leads to crackling noises when the flexible membrane 112 breaks contact with skin.

To maximise sound transfer to the second acoustic cavity 108, the flexible membrane 112 may have a Shore durometer as measured using ASTM D2240 type A of between 60 and 80, preferably between 70 and 80, for example 73.

In some embodiments, particularly when formed from silicone, the flexible membrane 112 has a thickness of between 0.4 mm and 0.8 mm, and preferably between 0.55 mm and 0.65 mm, for example 0.6 mm. In some embodiments, particularly when formed from a biaxially-oriented polyethylene terephthalate (e.g. Mylar®), the flexible membrane 112 may have a thickness of between 0.05 mm and 0.10 mm, for example 0.07 mm. In some embodiments, particularly when formed from a glass-reinforced epoxy laminate, the flexible membrane 112 may have a thickness of between 0.08 mm and 0.12 mm, for example 0.10 mm.

The inventors have found that the above preferred materials and dimensions of the flexible membrane 112 can lead to a flatter frequency response of the signal acquired by the MEMS microphone 106 over a wider frequency range. Specifically, the inventors have realised that providing the flexible membrane 112 formed from silicone of the specified durometer and thickness leads to a highly effective skin interface with minimized crackling when transitioning contact with the surface of skin, whilst exhibiting excellent frequency response over larger frequency ranges, due to the relatively high mass and low rigidity of silicone.

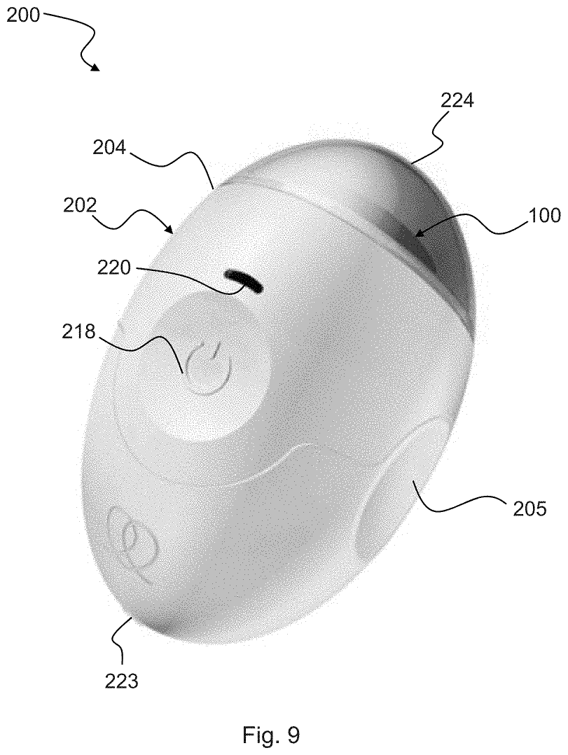

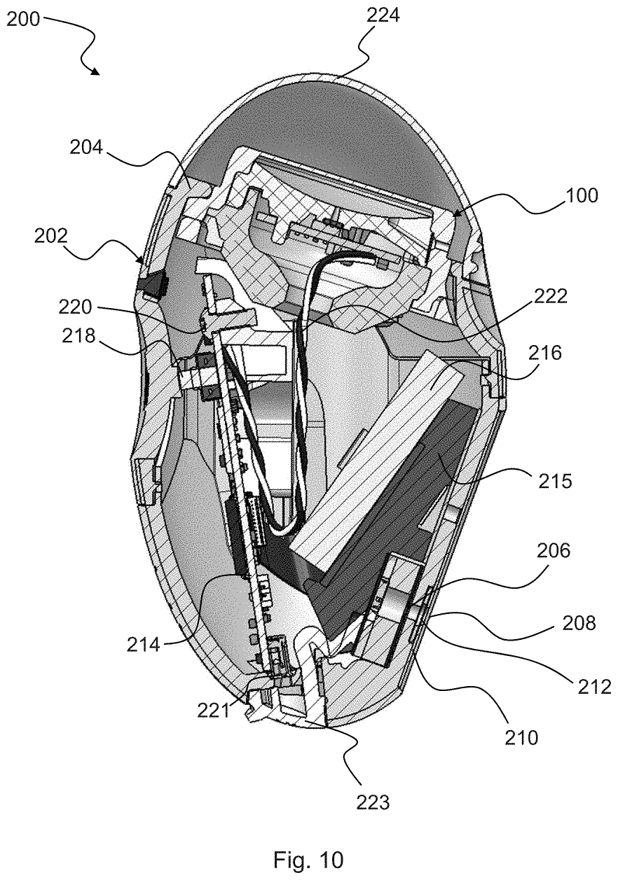

FIG. 9 and FIG. 10 provide perspective and cross-sectional views of a device 200 incorporating the apparatus 100 described above with reference to FIGS. 1 to 3. The device 200 comprises a sealed enclosure 202 encapsulating a portion of the apparatus 100 at a first open end 204 of the enclosure 202, the flexible membrane 112 of the apparatus 100 protruding from the first open end 204 of the enclosure 202, covered by a removable dust cap 224. The dust cap 224 is provided to cover the flexible membrane 112 of the apparatus 100 when the device 200 is not in use. The dust cap 224 engages with the open end 204 of the enclosure 202 in a push-fit configuration such that when engaged with the enclosure 202, the surface of the dust cap 224 abutting the enclosure 202 is flush with the surface of the enclosure 202 as shown in FIG. 9.

The device 200 is shaped to be held in the hand of a user in use. As such, a pair of indentations 205 are provided on either side of the enclosure 202 for thumb and forefinger of the user.

Shown in FIG. 10, within the housing 202 the device 200 further comprises a background microphone 206, which may be a MEMS microphone similar to the MEMS microphone 106 of the apparatus 100. An acoustic port 208 is provided in a side wall 210 of the enclosure 202 providing an acoustic path between the background microphone 206 and the exterior of the enclosure 202. The background microphone 206 is provided to pick up ambient sound for removal of ambient components also picked up by the MEMS microphone 106 of the apparatus 100. Optionally, a filter 212 may be provided in the acoustic path provided by the acoustic port 208 to mitigate ingress of dirt and/or moisture. The filter 212 may be similar to the optional filter 134 of the apparatus 100.

To further aid in the ergonomics of the device 200, a counter weight 215 may be provided within the enclosure 202 to counter the weight of the damping mass 136 of the apparatus 100 to ensure the centre of mass of the device 200 is proximal to the centre of the device 200.

The device 200 may further comprise a main printed circuit board (PCB) 214, a battery 216 an on/off button 218, a light indicator 220 such as a light emitting diode (LED), and a charging port 221. The on/off button 218, light indicator 220 and/or charging port may be mounted on the main PCB 214. An aperture is provided through the wall 210 of the enclosure 202 such that the charging port 221 can be accessed from the outside of the enclosure 202. A removable dust cover 223 may be provided to plug the aperture when the charging port 221 is not being used.

The main PCB 214 may have mounted thereon one or more processors, memory and an input/output (I/O) bus communicatively coupled with the processing circuitry and memory. The one or more processors may be operable to process signals received from the apparatus 100, via a cable harness 222 extending between the PCB 124 through the port 138 to the main PCB 214, and the second microphone 206. The main PCB 214 may additionally comprise circuitry for wired or wireless communication, e.g. Wi-Fi® or Bluetooth®, to allow audio signals acquired by the MEMS microphone 106 and/or the second microphone 206 to be transmitted to an auxiliary device, such as a smartphone, a computer, a tablet, or the like, or, indirectly, to the cloud. Methods for performing such techniques are known in the art and so will not be described in more detail here.

FIG. 11A is a cross-section of an apparatus 1100 of an example embodiment with a through-vent. Many of the components are the same as those shown in FIG. 2. As with FIG. 2, the housing 102 defines a first acoustic cavity which houses a MEMS microphone (not shown in FIG. 11A). The apparatus 1100 further comprises a second acoustic cavity 1108 defined by a cavity wall 1110, which separates the second acoustic cavity 1108 from the microphone housing 102. A through-vent 1180 is provided in the form of a circular hole extending through the cavity wall 1110. As shown by arrows 1190 in FIG. 11B, the through-vent 1180 provides an air path through cavity wall and into the interior of the apparatus via port 138.

It should be understood—especially by those having ordinary skill in the art with the benefit of this disclosure—that that the various operations described herein, particularly in connection with the figures, may be implemented by other circuitry or other hardware components. The order in which each operation of a given method is performed may be changed, and various elements of the devices illustrated herein may be added, reordered, combined, omitted, modified, etc. It is intended that this disclosure embrace all such modifications and changes and, accordingly, the above description should be regarded in an illustrative rather than a restrictive sense.

Similarly, although this disclosure makes reference to specific embodiments, certain modifications and changes can be made to those embodiments without departing from the scope and coverage of this disclosure. Moreover, any benefits, advantages, or solutions to problems that are described herein with regard to specific embodiments are not intended to be construed as a critical, required, or essential feature or element.

Further embodiments and implementations likewise, with the benefit of this disclosure, will be apparent to those having ordinary skill in the art, and such embodiments should be deemed as being encompassed herein. Further, those having ordinary skill in the art will recognize that various equivalent techniques may be applied in lieu of, or in conjunction with, the discussed embodiments, and all such equivalents should be deemed as being encompassed by the present disclosure.

It should be noted that the above-mentioned embodiments illustrate rather than limit the invention, and that those skilled in the art will be able to design many alternative embodiments without departing from the scope of the appended claims or embodiments. The word “comprising” does not exclude the presence of elements or steps other than those listed in a claim or embodiment, “a” or “an” does not exclude a plurality, and a single feature or other unit may fulfil the functions of several units recited in the claims or embodiments. Any reference numerals or labels in the claims or embodiments shall not be construed so as to limit their scope.

1. A contact sensor for monitoring breathing of a subject, comprising:
a microphone housing defining a first acoustic cavity;
a MEMS microphone disposed within the first acoustic cavity;
a second acoustic cavity separated from the first acoustic cavity by a cavity wall having a front surface and a rear surface, the second acoustic cavity at least partially defined by the front surface of the cavity wall;
an acoustic conduit formed between the first acoustic cavity and the second acoustic cavity through the cavity wall; and
a pressure relief vent having a first end terminating at the second acoustic cavity and a second end terminating outside of the second acoustic cavity.

2. The contact sensor of claim 1, wherein the cavity wall comprises a contact surface extending around an outer limit of the front surface cavity wall, wherein the contact surface is configured, in use, to contact the surface of the subject.

3. The contact sensor of claim 1, wherein the pressure relief vent is configured to vent air between the second acoustic cavity and the atmosphere.

4. The contact sensor of claim 1, further comprising a flexible membrane formed over the cavity wall, the flexible membrane having a front surface and a rear surface facing the front surface of the cavity wall.

5. The contact sensor of claim 1, wherein the pressure relief vent comprises a notch formed in the front surface of the cavity wall terminating at an outer limit of the cavity wall.

6. The contact sensor of claim 4,
wherein the pressure relief vent comprises a notch formed in the front surface of the cavity wall terminating at an outer limit of the cavity wall, and
wherein the flexible membrane is part of a cover, and wherein the pressure relief vent further comprises a side vent in the cover in fluid communication with the notch.

7. The contact sensor of claim 1, wherein the pressure relief vent comprises a passage formed between the front surface of the cavity wall and the rear surface of the cavity wall.

8. The contact sensor of claim 4, further comprising a gasket between the rear surface of the flexible membrane and the front surface of the cavity wall extending around an outer limit of the cavity wall, and wherein the pressure relief vent comprises a notch formed in the gasket.

9. The contact sensor of claim 1, further comprising a membrane filter disposed proximate the second end of the vent.

10-11. (canceled)

12. The contact sensor of claim 1, wherein the acoustic conduit terminates at a location approximately at the centre of the front surface of the cavity wall.

13. The contact sensor of claim 1, wherein the acoustic conduit has a diameter of approximately 0.5 mm and/or a length of between 0.5 mm and 5.0 mm.

14-17. (canceled)

18. The contact sensor of claim 4, wherein the flexible membrane comprises biaxially-oriented polyethylene terephthalate (Mylar®) or glass-reinforced epoxy laminate.

19. (canceled)

20. The contact sensor of claim 4, wherein the flexible membrane has a Shore durometer as measured using ASTM D2240 type A of between 60 and 80.

21. (canceled)

22. The contact sensor of claim 1, further comprising a damping mass coupled to the microphone housing.

23. (canceled)

24. The contact sensor of claim 1, comprising a printed circuit board (PCB), wherein the MEMS microphone is mounted on the PCB.

25. The contact sensor of claim 24, wherein the rear surface of the cavity wall comprises a plurality of pins extending therefrom, and wherein the PCB comprises a plurality apertures formed therethrough, each of the plurality of pins configured to engage with a respective aperture of the plurality of apertures so as to align the microphone housing with the acoustic conduit.

26. The contact sensor of claim 24, wherein an acoustic aperture is formed in the microphone housing and wherein upon engagement of the plurality of pins with the plurality of apertures, the acoustic aperture is aligned with the acoustic conduit.

27-28. (canceled)

29. A device, comprising: an enclosure;
the contact sensor of any one of the preceding claims; and
a background microphone configured to receive ambient sound,
wherein the contact microphone and the background microphone are housed in the enclosure.

30. The device of claim 29, wherein the MEMS microphone and the background microphone are substantially acoustically decoupled.

31. The device of claim 29, further comprising one or more acoustic dampeners housed in the enclosure.

32. (canceled)
